SEQUENCE LISTING (22 sequences)

SEQ ID NO: 1 - RNA, 20 nt, Homo sapiens
  source 1..20: mol_type = mRNA
tttggctcgggacttcaaaa

SEQ ID NO: 2 - RNA, 20 nt, Homo sapiens
  source 1..20: mol_type = mRNA
atttcatctttccaaattga

SEQ ID NO: 3 - RNA, 20 nt, Homo sapiens
  source 1..20: mol_type = mRNA
ttttgaagtcccgagccaaa

SEQ ID NO: 4 - RNA, 20 nt, Homo sapiens
  source 1..20: mol_type = mRNA
tcaatttggaaagatgaaat

SEQ ID NO: 5 - RNA, 20 nt, Homo sapiens
  source 1..20: mol_type = mRNA
tcccatcactgattttgaag

SEQ ID NO: 6 - RNA, 20 nt, Homo sapiens
  source 1..20: mol_type = mRNA
atcactgattttgaagtccc

SEQ ID NO: 7 - RNA, 20 nt, Homo sapiens
  source 1..20: mol_type = mRNA
tgattttgaagtcccgagcc

SEQ ID NO: 8 - RNA, 20 nt, Homo sapiens
  source 1..20: mol_type = mRNA
ttagccctaaagtcccaggt

SEQ ID NO: 9 - RNA, 20 nt, Homo sapiens
  source 1..20: mol_type = mRNA
ccaagaggcacaagtcctta

SEQ ID NO: 10 - RNA, 20 nt, Homo sapiens
  source 1..20: mol_type = mRNA
cagacaaatccaactacaac

SEQ ID NO: 11 - RNA, 20 nt, Homo sapiens
  source 1..20: mol_type = mRNA
atttcaagatacatgcgtcc

SEQ ID NO: 12 - RNA, 20 nt, Homo sapiens
  source 1..20: mol_type = mRNA
tgaagtcaatttaaatggaa

SEQ ID NO: 13 - RNA, 20 nt, Homo sapiens
  source 1..20: mol_type = mRNA
tttaaatggaactattgata

SEQ ID NO: 14 - RNA, 20 nt, Homo sapiens
  source 1..20: mol_type = mRNA
tggaactattgataaagtga

SEQ ID NO: 15 - RNA, 20 nt, Homo sapiens
  source 1..20: mol_type = mRNA
aagaaatcatgggacttgca

SEQ ID NO: 16 - RNA, 14 nt, Homo sapiens
  source 1..14: mol_type = mRNA
acaaaagcaggtta

SEQ ID NO: 17 - RNA, 15 nt, Homo sapiens
  source 1..15: mol_type = mRNA
gggggattgtcctca

SEQ ID NO: 18 - RNA, 17 nt, Homo sapiens
  source 1..17: mol_type = mRNA
ctatctagcccacccaa

SEQ ID NO: 19 - RNA, 15 nt, Homo sapiens
  source 1..15: mol_type = mRNA
gacattcacagacag

SEQ ID NO: 20 - RNA, 18 nt, Homo sapiens
  source 1..18: mol_type = mRNA
atcatttgttaaaacaca

SEQ ID NO: 21 - RNA, 18 nt, Homo sapiens
  source 1..18: mol_type = mRNA
caatttggaaagatgaaa

SEQ ID NO: 22 - RNA, 16 nt, Homo sapiens
  source 1..16: mol_type = mRNA
aatttggaaagatgaa

(19) United States
(12) Patent Application Publication
(10) Pub. No.: US 2025/0304958 A1
(43) Pub. Date: Oct. 2, 2025

(54) METHOD AND MOLECULES FOR REDUCING AXONAL TAU PROTEIN ACCUMULATION THROUGH BLOCKING OF HNRNP R-MEDIATED MAPT MRNA TRANSPORT FOR TREATMENT OF ALZHEIMER'S DISEASE

(71) Applicant: JULIUS-MAXIMILIANS-UNIVERSITÄT WÜRZBURG, Würzburg (DE)

(72) Inventors: Michael Anton SENDTNER, Würzburg (DE); Michael BRIESE, Würzburg (DE); Abdolhossein ZARE, Würzburg (DE); Saeede SALEHI, Würzburg (DE)

(73) Assignee: JULIUS-MAXIMILIANS-UNIVERSITÄT WÜRZBURG, Würzburg (DE)

(21) Appl. No.: 18/865,204

(22) PCT Filed: Mar. 13, 2023
(86) PCT No.: PCT/EP2023/056392

§ 371 Date: Nov. 12, 2024

Related U.S. Application Data
(60) Provisional application No. 63/364,629, filed on May 13, 2022.
(60) Provisional application No. 63/382,536, filed on Nov. 7, 2022.

(51) Int. Cl. C12N 15/113; A61P 25/28

(52) U.S. Cl. CPC C12N 15/113; A61P 25/28; C12N 2310/11

(57) ABSTRACT

This application includes methods for reducing axonal tau protein. Those methods include inhibiting the binding between MAPT mRNA and hnRNP R.

### CROSS-REFERENCE TO RELATED APPLICATIONS

This application claims priority to: (i) U.S. Provisional Patent Application No. 63/364,629, filed May 13, 2022; and (ii) U.S. Provisional Patent Application No. 63/382,536, filed Nov. 7, 2022; the entire contents of all of which are incorporated by reference herein.

### FIELD OF THE INVENTION

The present disclosure relates generally to a method for selective reduction of tau in axons by preventing the transport of the Microtubule-associated protein tau (MAPT) mRNA encoding tau protein from the cell body into axons by blocking the interaction of MAPT mRNA with hnRNP R, or by reducing hnRNP R levels. hnRNP R is an RNA-binding protein that interacts with the 3′UTR of MAPT mRNA. Molecules that inhibit such interaction between MAPT mRNA and hnRNP R, or that lower hnRNP R levels, reduce axonal tau protein.

### BACKGROUND

Alzheimer's disease (AD) is a neurodegenerative disorder and the most common form of late-onset dementia, affecting a substantial proportion of individuals aged 65 and over. It is characterized by progressive memory loss and is expected to increase dramatically over the coming decades as aging is the main risk factor. AD is caused by the accumulation of insoluble protein aggregates in the brain, including the formation of tau fibrils in neuronal axons, leading to neuron dysfunction and loss, which in turn results in progressive memory loss leading to a reduced ability to execute daily functions.

Treating AD is challenging because of the disease's complex etiology. In symptomatic AD patients, two types of protein aggregates are present in the brain: extracellular accumulations of Amyloid-β (Aβ) protein (senile plaques, SPs) and intracellular fibrils of hyperphosphorylated tau protein (neurofibrillary tangles, NFTs). Even though SPs are present widespread in brains of AD patients, the temporal and spatial formation of NFTs correlates more closely with the cognitive impairments and the progression of the disease. While most therapeutic approaches have focused on removing or delaying the formation of SPs, it is increasingly clear that preventing the formation of NFTs or halting their spread offers to be a promising therapeutic option. Additionally, current therapeutic strategies aimed at preventing or slowing down the formation of plaques and tangles through antibody-based targeting of Aβ or tau may induce unwanted side-effects.

NFTs initially occur in the entorhinal cortex and the hippocampus, the site of memory formation affected first in AD, whereas SPs arise more diffusely throughout the brain. Furthermore, axon dysfunction is an early event in AD inducing neuronal degeneration through “dying back” mechanisms spreading from the damaged axons to neuronal cell bodies. Salvadores, N., et al., Axonal Degeneration in AD: The Contribution of Abeta and Tau. Front Aging Neurosci, 2020. 12: p. 581767.

Tau is needed in the brain for axon maintenance by stabilizing the cytoskeleton through microtubule assembly. This function is impaired by hyperphosphorylation of tau leading to its fibrillization and toxic accumulation as NFTs in axons. This results in axonal tau aggregates, which develop early during AD and disrupt transport of RNAs and proteins required for axon and synapse maintenance. Robbins, M., et al., Synaptic tau: A pathological or physiological phenomenon? Acta Neuropathol Commun, 2021. 9(1): p. 149. This suggests that NFT formation is a critical step in the etiology underlying AD. Therefore, reducing tau and thus tau aggregates appears promising, as genetic deletion of tau improved cognitive performance in a mouse model of AD, even without affecting the deposition of SPs. Roberson, E.D., et al., Reducing endogenous tau ameliorates amyloid beta-induced deficits in an Alzheimer's disease mouse model. Science, 2007. 316 (5825): p. 750-4.

In the hippocampus of AD patients, axonal tau pathology precedes tau deposition in somatodendritic regions suggesting that dysregulation of axonal tau alterations is an early event in the pathological AD cascade. Christensen, K.R., et al., Pathogenic tau modifications occur in axons before the somatodendritic compartment in mossy fiber and Schaffer collateral pathways. Acta Neuropathol Commun, 2019. 7(1): p. 29. Beyond that, tau is secreted upon neuronal activation through interaction with synaptic vesicle proteins, facilitating spreading of tau pathology between brain regions in AD. de Calignon, A., et al., Propagation of tau pathology in a model of early Alzheimer's disease. Neuron, 2012. 73(4): p. 685-97. Tau reduction improves phenotypic defects of AD mouse models and restores axonal transport defects induced by A≈, indicating a causal role of tau in AD pathogenesis. Leroy, K., et al., Lack of tau proteins rescues neuronal cell death and decreases amyloidogenic processing of APP in APP/PS1 mice. Am J Pathol, 2012. 181(6): p. 1928-40.

Strategies for tau removal can be broadly classified into two approaches. In the first approach, tau is reduced globally-MAPT mRNA levels are reduced in a targeted manner through delivery of short double-stranded RNAs in the form of short interfering RNAs (siRNAs) or short hairpin RNAs (shRNAs), or through delivery of antisense oligonucleotides (ASOs) that elicit RNase H-mediated mRNA degradation. However, because tau is involved in regulating gene expression, its non-specific loss would affect many other pathways. Montalbano, M., et al., Tau Modulates mRNA Transcription, Alternative Polyadenylation Profiles of hnRNPs, Chromatin Remodeling and Spliceosome Complexes. Front Mol Neurosci, 2021. 14: p. 742790. Further, depletion of tau throughout neurons is detrimental to their functioning as it is involved in several processes related to synaptic plasticity. Accordingly, tau knockout mice exhibit cognitive defects and impaired motor performance. Lei, P., et al., Motor and cognitive deficits in aged tau knockout mice in two background strains. Mol Neurodegener, 2014. 9: p. 29. Additionally, acute knockdown of tau bilaterally in the hippocampus of mice caused defects in motor coordination and spatial memory. Velazquez, R., et al., Acute tau knockdown in the hippocampus of adult mice causes learning and memory deficits. Aging Cell, 2018. 17(4): p. e12775. Thus, global reduction of tau protein is not an effective strategy because the removal of NFTs that might be achieved through this approach would be accompanied by unwanted alterations in those cellular functions that are normally carried out by tau in the cell body of neurons.

In the second approach, tau-specific antibodies are used that neutralize and/or remove pathological tau. Jadhav, S., et al., A walk through tau therapeutic strategies. Acta Neuropathol Commun, 2019. 7(1): p. 22. To that end, different antibodies have been developed that are specific for tau's hyperphosphorylated form and that target various regions of tau. However, while antibody-based strategies have shown some success by preventing tau seeding and the formation of NFTs, the strategies are limited by the occurrence of multiple tau isoforms and pathological fragments that might not be targeted simultaneously with individual antibodies. Further, antibody delivery to the brain is inefficient and may require repeated administration. Additionally, immunization against targets in the brain, whether active or passive, might induce inflammatory cascades causing further complications and leading to acute disease states.

Also, adverse effects often accompany currently available therapeutic strategies. For example, immunotherapies that utilize antibodies targeting AB or tau to induce clearing of SPs and NFTs by the immune system might trigger inflammatory responses, cerebral edemas or hemorrhages as side effects. Other approaches aim to reduce levels of the tau protein through antisense oligonucleotide (ASO)-mediated degradation of the MAPT transcript encoding tau. DeVos, S. L., et al., Tau reduction prevents neuronal loss and reverses pathological tau deposition and seeding in mice with tauopathy. Sci Transl Med, 2017. 9(374): p. eaag0481; Easton, A., et al., Identification and characterization of a MAPT-targeting locked nucleic acid antisense oligonucleotide therapeutic for tauopathies. Mol Ther Nucleic Acids, 2022. 29: p. 625-42.

An additional challenge for Aβ and tau immunotherapies is to identify the isoform and aggregate species that needs to be targeted in order to achieve a therapeutic outcome. Song, C., et al., Immunotherapy for Alzheimer's disease: targeting beta-amyloid and beyond. Transl Neurodegener, 2022. 11(1): p. 18. Both Aβ and tau exist as fragments of different length or splice isoforms, respectively, and their aggregation progresses from an oligomeric state towards fibrillary deposits. Thus, beyond the currently available strategies of targeting selected Aβ and tau deposits, it is desired to identify additional factors that regulate SP and NFT abundance and whose manipulation can be tolerated.

Therefore, there is a need for a new method whereby tau concentration is selectively decreased from the axons of neurons. One such method can include an inhibition, reduction and/or depletion of the RNA-binding protein hnRNP R, which may lead to a reduction in plaques and tangles.

### SUMMARY

The present invention discloses a method for preventing the transport of the Microtubule-associated protein tau (MAPT) mRNA encoding tau protein from the cell body into axons. By blocking the interaction of MAPT mRNA with hnRNP R, or by reducing the levels of hnRNP R, an RNA-binding protein that interacts with the 3′UTR of MAPT mRNA and facilitates its axonal localization, reduced MAPT mRNA transport occurs. As a result of reduced mRNA transport, local tau protein synthesis in axons is decreased and lower tau protein levels selectively in axons are achieved, retaining the tau levels in the somatodendritic compartment. The reduced availability of newly synthesized axonal tau protein limits the levels of tau protein that can be hyperphosphorylated and transsynaptically transmitted, thereby reducing the formation of NFTs and the spreading of tau pathology. This should interfere with the progression of AD. The method allows tau to continue to function in neuronal cell bodies, while axonal NFT formation is blocked. Preventing the local accumulation of tau in axons achieves a more specific removal of tau aggregates, with fewer side-effects than the prior art methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows results of individual nucleotide resolution crosslinking and immunoprecipitation (iCLIP).

FIG. 1B shows RNA co-immunoprecipitation results.

FIG. 1C shows compartmentalized cultures of mouse motoneurons.

FIG. 1D shows quantitative PCR (qPCR) results.

FIG. 2A shows fluorescent in situ hybridization (FISH).

FIG. 2B shows quantification of the FISH signal.

FIG. 3A shows immunostaining of motoneurons.

FIG. 3B shows quantification of tau immunosignals.

FIG. 4 depicts the creation and accumulation of tau fibrils in AD and compares it against the proposed mechanism/method of the present disclosure to reduce tau pathology in AD.

FIG. 5 shows the design of antisense oligonucleotides (ASOs).

FIGS. 6A-6B demonstrate that hnRNP R binds to Mapt mRNA. FIG. 6A includes UCSC genome browser views showing hnRNP R binding sites along the Mapt pre-mRNA revealed by individual nucleotide resolution crosslinking and immunoprecipitation (iCLIP). FIG. 6B is a graphical illustration of the results of co-immunoprecipitation of Mapt mRNA with an anti-hnRNP R antibody from mouse motoneurons at DIV 7. Data are mean±standard deviation (s.d.) of n=5 independent experiments.

FIGS. 7A-7D demonstrate that reduction in hnRNP R function reduces Mapt mRNA levels in axons of motoneurons. FIG. 7A is a number of images of fluorescent in situ hybridization (FISH) of Mapt mRNA in motoneurons cultured from Hnrnpr+/+ and −/− mice at DIV 5. Scale bars: 10 μm and 5 μm (inset). FIG. 7B is a graphical illustration, quantifying the Mapt FISH signal in axons. Statistical analysis was performed using a Mann-Whitney test (Hnrnpr+/+, N=78 motoneurons; Hnrnpr−/−, N=70; n=3 independent experiments). Data are shown as Tukey box plots. FIG. 7C is a schematic of a microfluidic chamber for compartmentalized neuron cultures. FIG. 7D is a graphical illustration of the results of quantitative PCR of Mapt mRNA from somatodendritic and axonal RNA of Hnrnpr+/+ and −/− mouse motoneurons at DIV 7. Statistical analysis was performed using a two-tailed one-sample t-test. Data are mean±s.d. of n=3 independent experiments.

FIGS. 8A-8C demonstrate that reduction in hnRNP R function reduces tau protein levels in axons of motoneurons. FIG. 8A is several images of tau immunostaining of motoneurons cultured from Hnrnpr+/+ and −/− mice at DIV 5, with proximal and distal regions of the axon marked. GFP expression was used for visualization of neuronal morphology and for normalization of tau levels. Scale bars: 10 μm and 5 μm (inset). FIGS. 8B and 8C are graphical illustrations of the quantification of the tau (FIG. 8B) and Tubulin (FIG. 8C) immunosignal in the somata and proximal and distal axonal regions of motoneurons from Hnrnpr+/+ and −/− mice (Hnrnpr+/+, N=48 motoneurons; Hnrnpr−/−, N=42; n=3 independent experiments). Statistical analysis was performed using a Mann-Whitney test. Data are shown as Tukey box plots.

FIG. 9 is a representation of the design of two antisense oligonucleotides (MAPT-ASO1 and MAPT-ASO2) for blocking the interaction between hnRNP R and Mapt mRNA.

FIGS. 10A-10B demonstrate oligonucleotide uptake in motoneurons and hippocampal neurons. FIG. 10A are immunofluorescence images of mouse motoneurons (MN) treated with different concentrations of a Cy3-labeled sense oligonucleotide at DIV 6. Scale bars: 10 μm and 5 μm (inset). FIG. 10B are immunofluorescence images of untreated (Ctrl) mouse hippocampal neurons (HN), and hippocampal neurons treated with 10 μM of a Cy3-labeled sense oligonucleotide at DIV 25. Scale bars: 10 μm and 5 μm (inset).

FIGS. 11A-F demonstrate that treatment with MAPT-ASO1 and -ASO2 reduces axonal Mapt mRNA levels. FIG. 11A is images of Mapt FISH in untreated (Ctrl) mouse motoneurons and motoneurons treated with MAPT-ASO1 or -ASO2 at DIV 6. Scale bars: 10 μm and 5 μm (inset). FIGS. 11B-11C are graphical representations quantifying the Mapt FISH signal in the somata (FIG. 11B) and axons (FIG. 11C) of motoneurons. Statistical analysis was performed using a Kruskal-Wallis test with Dunn's multiple comparisons test. (Ctrl, N=56 motoneurons; ASO1, N=46; ASO2, N=55; n=3 independent experiments). Data are shown as Tukey box plots. FIG. 11D is images of Mapt FISH in untreated (Ctrl) mouse hippocampal neurons and hippocampal neurons treated with MAPT-ASO1 or-ASO2 at DIV 6. Scale bars: 10 μm and 5 μm (inset). FIGS. 11E-11F are graphical representations, quantifying the Mapt FISH signal in the somata (FIG. 11E) and axons (FIG. 11F) of hippocampal neurons. Statistical analysis was performed using a Kruskal-Wallis test with Dunn's multiple comparisons test. (Ctrl, N=23 hippocampal neurons; ASO1, N=24; ASO2, N=23; n=3 independent experiments). Data are shown as Tukey box plots.

FIGS. 12A-12D demonstrate that treatment with MAPT-ASO1 and -ASO2 reduces axonal tau protein levels. FIG. 12A is images of tau immunostaining of untreated (Ctrl) mouse motoneurons and motoneurons treated with MAPT-ASO1 or -ASO2 at DIV 11, with proximal and distal regions of the axon marked. Scale bars: 10 μm and 5 μm (inset). FIGS. 12B-12D are graphical illustrations quantifying the tau immunosignal in the somata (FIG. 12B) and proximal (FIG. 12C) and distal (FIG. 12D) axonal regions of untreated and treated motoneurons. Statistical analysis was performed using a Kruskal-Wallis test with Dunn's multiple comparisons test. (Ctrl, N=43 motoneurons; ASO1, N=35; ASO2, N=39; n=3 independent experiments). Data are shown as Tukey box plots.

FIGS. 13A-13B demonstrate MAPT-ASO2 uptake in motoneurons and hippocampal neurons. FIG. 13A is immunofluorescence images of untreated (Ctrl) mouse motoneurons, and motoneurons treated with 10 μM of a Cy3-labeled scramble oligonucleotide as control or MAPT-ASO2 at DIV 6. Scale bars: 10 μm and 5 μm (inset). FIG. 13B is immunofluorescence images of untreated (Ctrl) mouse hippocampal neurons, and hippocampal neurons treated with 10 μM of a Cy3-labeled scramble oligonucleotide or MAPT-ASO2 at DIV 25. Scale bars: 10 μm and 5 μm (inset).

FIGS. 14A-14F demonstrate that treatment with MAPT-ASO2 reduces axonal Mapt mRNA levels relative to treatment with a scramble oligonucleotide. FIG. 14A is images of Mapt FISH in untreated (Ctrl) mouse motoneurons and motoneurons treated with scramble oligonucleotide or MAPT-ASO2 at DIV 6. Scale bars: 10 μm and 5 μm (inset). FIGS. 14B-14C are graphical illustrations quantifying the Mapt FISH signal in the somata (FIG. 14B) and axons (FIG. 14C) of motoneurons. Statistical analysis was performed using a Kruskal-Wallis test with Dunn's multiple comparisons test. (Ctrl, N=36 motoneurons; Scramble, N=46; ASO2, N=45; n=3 independent experiments). Data are shown as Tukey box plots. FIG. 14D is images of Mapt FISH in untreated (Ctrl) mouse hippocampal neurons and hippocampal neurons treated with scramble oligonucleotide or MAPT-ASO2 at DIV 6. Scale bars: 10 μm and 5 μm (inset). FIGS. 14E-14F are graphical illustrations quantifying the Mapt FISH signal in the somata (FIG. 14E) and axons (FIG. 14F) of hippocampal neurons. Statistical analysis was performed using a Kruskal-Wallis test with Dunn's multiple comparisons test. (Ctrl, N=50 hippocampal neurons; Scramble, N=46; ASO2, N=47; n=3 independent experiments). Data are shown as Tukey box plots.

FIGS. 15A-15C demonstrate a reduced tau synthesis in axons of neurons treated with MAPT-ASO2. FIG. 15A is images of newly synthesized tau protein in untreated (Ctrl) mouse motoneurons and motoneurons treated with scramble oligonucleotide or MAPT-ASO2 using a puromycin labeling with proximity ligation assay (Puro-PLA) at DIV 6. Scale bars: 10 μm and 5 μm (inset). FIGS. 15B and 15C are graphical illustrations quantifying the tau Puro-PLA signal in the somata (FIG. 15B) and axons (FIG. 15C) of motoneurons. Statistical analysis was performed using a Kruskal-Wallis test with Dunn's multiple comparisons test. (Ctrl, N=9 motoneurons; Scramble, N=24; ASO2, N=29; n=2 independent experiments). Data are shown as Tukey box plots.

FIGS. 16A-16G demonstrate that treatment with MAPT-ASO2 reduces axonal tau protein levels relative to treatment with a scramble oligonucleotide. FIG. 16A is images of tau immunostaining of untreated (Ctrl) mouse motoneurons and motoneurons treated with scramble oligonucleotide or MAPT-ASO2 at DIV 11, with proximal and distal regions of the axon marked. Scale bars: 10 μm and 5 μm (inset). FIGS. 16B-16D are graphical illustrations quantifying the tau immunosignal in the somata (FIG. 16B) and proximal (FIG. 16C) and distal (FIG. 16D) axonal regions of motoneurons. Statistical analysis was performed using a Kruskal-Wallis test with Dunn's multiple comparisons test. (Ctrl, N=54 motoneurons; Scramble, N=65; ASO2, N=62; n=3 independent experiments). Data are shown as Tukey box plots. FIG. 16E is images of tau immunostaining of untreated (Ctrl) mouse hippocampal neurons and hippocampal neurons treated with scramble oligonucleotide or MAPT-ASO2 at DIV 25, with distal regions of the axon marked. Scale bars: 10 μm and 5 μm (inset). FIGS. 16F-16G are graphical illustrations quantifying the tau immunosignal in the somata (FIG. 16F) and distal axonal regions (FIG. 16G) of hippocampal neurons. Statistical analysis was performed using a Kruskal-Wallis test with Dunn's multiple comparisons test. (Ctrl, N=38 hippocampal neurons; Scramble, N=36; ASO2, N=42; n=3 independent experiments). Data are shown as Tukey box plots.

FIGS. 17A-17E demonstrate that treatment with MAPT-ASO2 reduces axon growth relative to treatment with a scramble oligonucleotide. FIG. 17A is images showing the morphology of untreated (Ctrl) mouse motoneurons and motoneurons treated with scramble oligonucleotide or MAPT-ASO2 at DIV 7. Scale bar: 50 μm. FIG. 17B is a graphical illustration quantifying axon lengths of motoneurons. Statistical analysis was performed using a Kruskal-Wallis test. (Ctrl, N=195 motoneurons; Scramble, N=254; ASO2, N=234; n=3 independent experiments). Data are shown as Tukey box plots. FIG. 17C is a graphical illustration quantifying motoneuron survival. Statistical analysis was performed using one-way ANOVA. Data are mean±s.d. of n=4-6 independent experiments. FIG. 17D is images showing the morphology of untreated (Ctrl) mouse hippocampal neurons and hippocampal neurons treated with scramble oligonucleotide or MAPT-ASO2 at DIV 25. Scale bar: 50 μm. FIG. 17E is a graphical illustration quantifying axon lengths of hippocampal neurons. Statistical analysis was performed using a Kruskal-Wallis test. (Ctrl, N=81 hippocampal neurons; Scramble, N=85; ASO2, N=77; n=3 independent experiments). Data are shown as Tukey box plots.

FIGS. 18A-18C demonstrate identification of additional ASOs for reducing axonal Mapt mRNA levels. FIG. 18A illustrates binding sites of MAPT-ASO1 to -ASO20 in the Mapt 3′ UTR. FIG. 18B illustrates sequences of MAPT-ASO1 to -ASO20 and their binding positions along human MAPT NCBI Reference Sequence NG_007398.2. Mapt mRNA levels were quantified by FISH in somata and axons of ASO-treated mouse hippocampal neurons and normalized to untreated hippocampal neurons. MAPT-ASOs with >50% reduction of axonal Mapt mRNA levels are highlighted. FIG. 18C illustrates the quantification of the Mapt FISH signal in the somata and axons of hippocampal neurons at DIV6. Data are shown as Tukey box plots.

FIG. 19 are images of depletion of hnRNP R reducing senile plaque load in Alzheimer's disease mice. Coronal brain sections of 5xFAD; Hnrnpr+/+ and 5xFAD; Hnrnpr−/− mice were immunostained with antibody 6E10 to label Aβ and an antibody against lba1 to label microglia. Nuclei were stained with 4′,6-diamidino-2-phenylindole (DAPI).

FIG. 20 are images of depletion of hnRNP R reducing phosphorylated tau in Alzheimer's disease mice. Coronal brain sections of 5xFAD; Hnrnpr+/+ and 5xFAD; Hnrnpr−/− mice were immunostained with antibody AT8 to label phosphorylated tau and an antibody against total tau. Nuclei were stained with DAPI.

FIG. 21 is an illustration of an additional proposed mechanism for the depletion of hnRNP R through antisense oligonucleotides (ASOs) targeting the HNRNPR mRNA or inhibition of hnRNP R through small molecules, peptides and/or oligonucleotides, leading to reduced amounts of senile plaques and neurofibrillary tangles.

### DETAILED DESCRIPTION

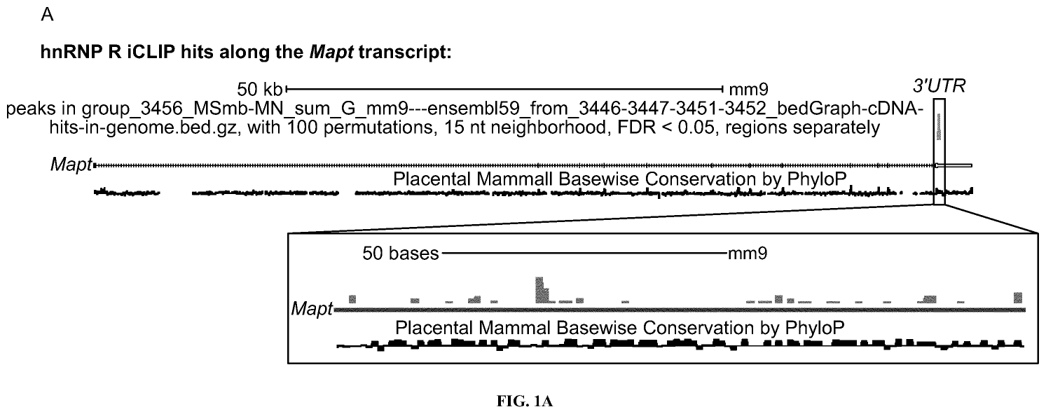

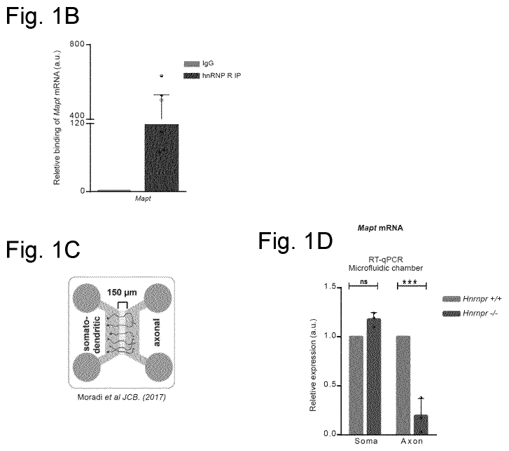

The claimed method is based in part on the finding that the RNA-binding protein hnRNP R interacts with the 3′UTR of Mapt mRNA in motoneurons and regulates its axonal localization, as shown in FIGS. 1A and 1B, which are further described below.

To facilitate the understanding of this disclosure a number of terms of in quotation marks are defined below. It is noted that the drawings of the present application are provided for illustrative purposes only and, as such, the drawings are not drawn to scale. It is also noted that like and corresponding elements are referred to by like reference numerals.

In the following description, numerous specific details are set forth, such as particular structures, components, materials, dimensions, processing steps and techniques, in order to provide an understanding of the various embodiments of the present application. However, it will be appreciated by one of ordinary skill in the art that various embodiments of the present application may be practiced without these specific details. In other instances, well-known structures or processing steps have not been described in detail in order to avoid obscuring the present application.

As used herein, the term “substantially” or “substantial”, is equally applicable when used in a negative connotation to refer to the complete or near complete lack of an action, characteristic, property, state, structure, item, or result. For example, a surface that is “substantially” flat would either be completely at, or so nearly flat that the effect would be the same as if it were completely flat.

As used herein, terms defined in the singular are intended to include those terms defined in the plural and vice versa.

As used in this specification and its appended claims, terms such as “a”, “an” and “the” are not intended to refer to only a singular entity, but include the general class of which a specific example may be used for illustration, unless the context dictates otherwise. The terminology herein is used to describe specific embodiments of the invention, but their usage does not delimit the invention, except as outlined in the claims.

Unless otherwise indicated, all numbers expressing quantities of ingredients, properties such as molecular weights, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

Thus, reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes whole numbers of 5, 6, 7, 8, 9, and 10, and fractional numbers 5.1, 5.2, 5.3, 5,4, 5,5, 5.6, 5.7, 5.8, 5.9, etc.

In the discussion and claims herein, the tern “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

In accordance with the method of the present disclosure, the disclosed inhibitory compounds may be administered for either a prophylactic or therapeutic purpose either alone or with other immunosuppressive or anti-inflammatory agents. When provided prophylactically, the immunosuppressive compound(s) are provided in advance of any inflammatory response or symptom (for example, prior to, at, or shortly after the time of an organ or tissue transplant but in advance of any symptoms of organ rejection).

The disclosed inhibitory compounds may, in accordance with the invention, be administered in single or divided doses by the oral, parenteral or topical routes. A suitable oral dosage for a compound of formula I would be in the range of about 0.001 mg to about 10 g per day. In parenteral formulations, a suitable dosage unit may contain from about 0.001 mg to about 250 mg of said compounds, whereas for topical administration, formulations containing about 0.001% to about 1% active ingredient are contemplated. It should be understood, however, that the dosage administration from patient to patient will vary and the dosage for any particular patient will depend upon the clinician's judgement, who will use as criteria for fixing a proper dosage the size and condition of the patient as well as the patient's response to the drug.

When the compounds of the present disclosure are to be administered by the oral route, they may be administered as medicaments in the form of pharmaceutical preparations which contain them in association with a compatible pharmaceutical carrier and/or excipient material. Such carrier and/or excipient material can be an inert organic or inorganic carrier and/or excipient material suitable for oral administration. Examples of such carrier and/or excipient materials are water, gelatin, talc, starch, magnesium stearate, gum arabic, vegetable oils, polyalkylene-glycols, petroleum jelly and the like.

The pharmaceutical preparations can be prepared in a conventional manner and finished dosage forms can be solid dosage forms, for example, tablets, dragees, capsules, and the like, or liquid dosage forms, for example solutions, suspensions, emulsions and the like. The pharmaceutical preparations may be subjected to conventional pharmaceutical operations such as sterilization. Further, the pharmaceutical preparations may contain conventional adjuvants such as preservatives, stabilizers, emulsifiers, flavor-improvers, wetting agents, buffers, salts for varying the osmotic pressure and the like. Solid carrier and/or excipient material which can be used include, for example, starch, lactose, mannitol, methyl cellulose, microcrystalline cellulose, talc, silica, dibasic calcium phosphate, and high molecular weight polymers (such as polyethylene glycol).

For parenteral use, the inhibitory compounds can be administered in an aqueous or non-aqueous solution, suspension or emulsion in a pharmaceutically acceptable oil or a mixture of liquids, which may contain bacteriostatic agents, antioxidants, preservatives, buffers or other solutes to render the solution isotonic with the blood, thickening agents, suspending agents or other pharmaceutically acceptable additives. Additives of this type include, for example, tartrate, citrate and acetate buffers, ethanol, propylene glycol, polyethylene glycol, complex formers (such as EDTA), antioxidants (such as sodium bisulfite, sodium metabisulfite, and ascorbic acid), high molecular weight polymers (such as liquid polyethylene oxides) for viscosity regulation and polyethylene derivatives of sorbitol anhydrides. Preservatives may also be added if necessary, such as benzoic acid, methyl or propyl paraben, benzalkonium chloride and other quaternary ammonium compounds.

The compounds of this disclosure may also be administered as solutions for nasal application and may contain in addition to the compounds of this invention suitable buffers, tonicity adjusters, microbial preservatives, antioxidants and viscosity-increasing agents in an aqueous vehicle. Examples of agents used to increase viscosity are polyvinyl alcohol, cellulose derivatives, polyvinylpyrrolidone, polysorbates or glycerin. Microbial preservatives added may include benzalkonium chloride, thimerosal, chloro-butanol or phenylethyl alcohol.

Additionally, the compounds provided in this disclosure can be administered topically or by suppository.

### EXAMPLES

### Example 1

An investigation of the functional significance of the interaction of the RNA-binding protein hnRNP R with the 3′UTR of Mapt mRNA in motoneurons was conducted. hnRNP R knockout mice were generated (Hnrnpr−/−) and their motoneurons were cultured in microfluidic chambers (FIG. 1C). Such compartmentalization of motoneurons allows separate extraction of RNA from their somatodendritic and axonal regions for analysis of transcript levels by quantitative PCR (qPCR). This showed that loss of hnRNP R depletes Mapt mRNA from axons but not from cell bodies including dendrites (FIG. 1D). This result suggests that hnRNP R mediates transport of Mapt mRNA into axons.

FIG. 1A shows hnRNP R binding sites along the Mapt pre-mRNA revealed by individual nucleotide resolution crosslinking and immunoprecipitation (iCLIP). FIG. 1B shows the co-immunoprecipitation of Mapt mRNA with an anti-hnRNP R antibody from mouse motoneurons. FIG. 1C is a schematic of microfluidic chamber for compartmentalized neuron cultures. FIG. 1D is a quantitative PCR of Mapt from somatodendritic and axonal RNA of Hnrnpr+/+ and −/− mouse motoneurons. A statistical analysis was performed using a two-way ANOVA with Sidak's multiple comparisons test. *** p<0.001.

Fluorescent in situ hybridization (FISH) for Mapt mRNA was then performed in cultured motoneurons from hnRNP R knockout and wildtype control mice. In line with the qPCR results from compartmentalized chambers, FISH revealed reduced Mapt mRNA amounts in axons of motoneurons depleted of hnRNP R.

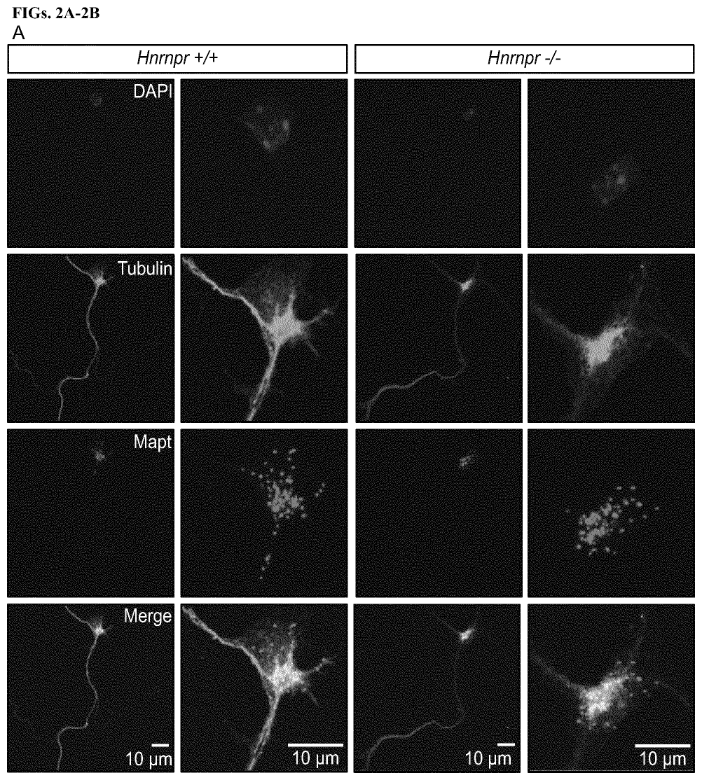

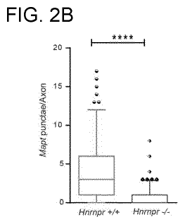

FIG. 2A depicts fluorescent in situ hybridization (FISH) results of Mapt mRNA in motoneurons cultured from Hnrnpr+/+ and −/− mice. FIG. 2B shows quantification of the FISH signal. A statistical analysis was performed using a Mann-Whitney test. **** p<0.0001.

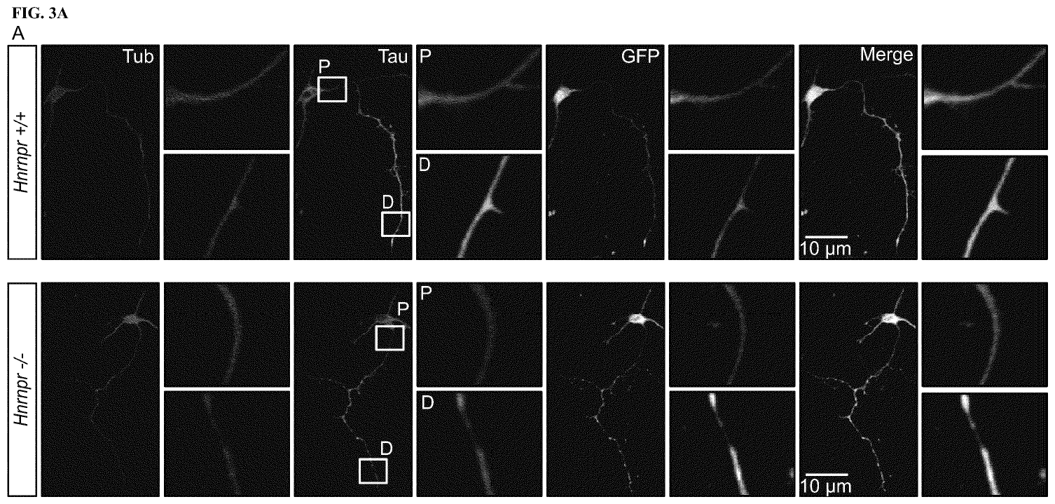

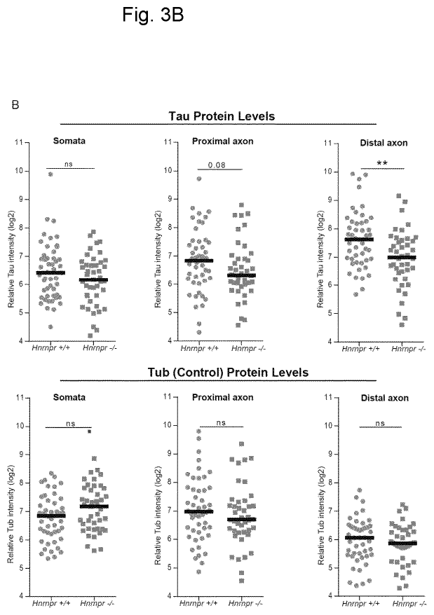

Finally, immunostaining showed that loss of hnRNP R leads to reduced levels of tau protein in axons, both in their proximal and distal regions, while its levels in the somata were unchanged. FIG. 3A shows tau immunostaining of motoneurons cultured from Hnrnpr+/+ and −/− mice, with proximal (P) and distal (D) regions of the axon marked. FIG. 3B is the quantification of tau immunosignal from the immunostaining. A statistical analysis was performed using a Mann-Whitney test. ** p<0.01; n.s., not significant.

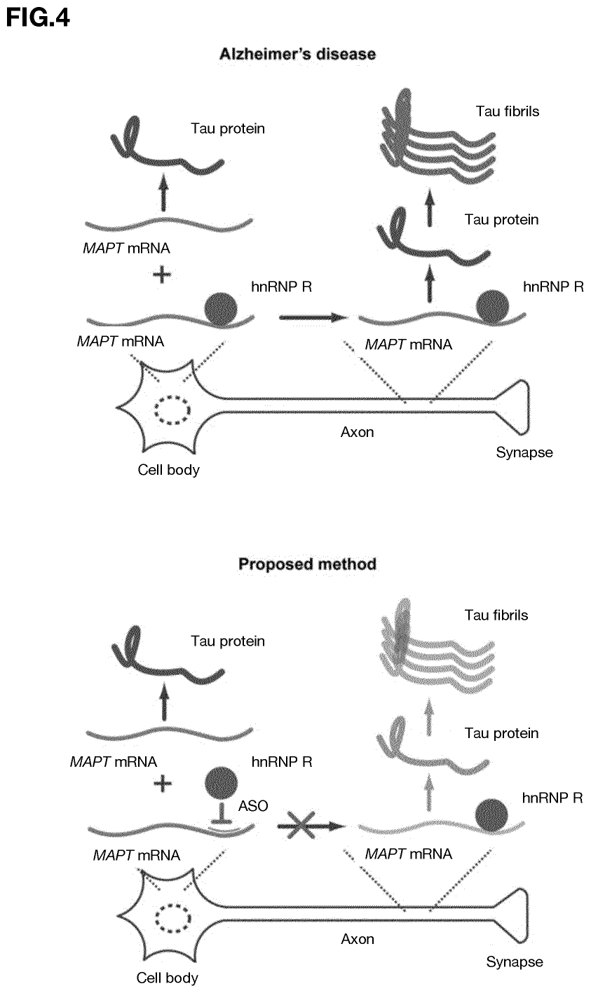

FIG. 4 (left panel) shows that in the diseased state, MAPT mRNA is transported into axons by hnRNP R where it is locally translated into tau protein, giving rise to NFTs.

Given that hnRNP R interacts with hundreds of RNAs including the abundant noncoding RNA 7SK, however, its widespread depletion could have detrimental side-effects. Therefore, the use of small molecules, peptides, ASOs or combinations thereof can be used to inhibit the association between MAPT 3′UTR and hnRNP R. These small molecules, peptides, ASOs or combinations thereof anneal to the region of the MAPT 3′UTR which hnRNP R otherwise binds to, in order to block the interaction between hnRNP R with MAPT. As a result of these treatments, transport of MAPT into axons and local synthesis of tau protein should be reduced, thereby alleviating tau pathology.

FIG. 4 (right panel) shows a proposed mechanism of the invention, in which ASOs are being used to block hnRNP R from binding to MAPT mRNA, thereby preventing its axonal localization and local synthesis of tau. As a result, formation of tau fibrils is reduced.

ASOs have been successfully delivered to the central nervous system and used therapeutically to correct the splicing defect underlying the motoneuron disorder spinal muscular atrophy. Jablonka, S. and M. Sendtner, Developmental regulation of SMN expression: pathophysiological implications and perspectives for therapy development in spinal muscular atrophy. Gene Ther, 2017. 24(9): p. 06-513.

If the two binding sites of the MAPT mRNA are bound by either a small molecule, peptide, or ASO, the mRNA binding is blocked. Two mRNA sites associated with the hnRNP R binding protein are: MAPT-S1: 5′-TTTGGCTCGGGACTTCAAAA-3′ and MAPT-S2: 5′-ATTTCATCTTTCCAAATTGA-3′.

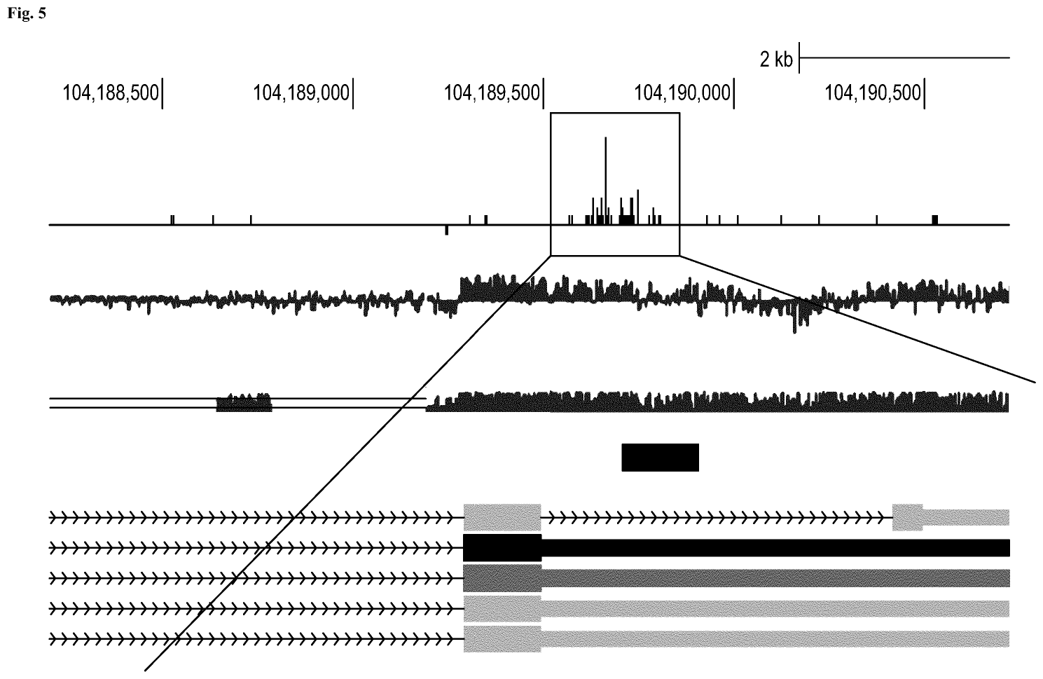

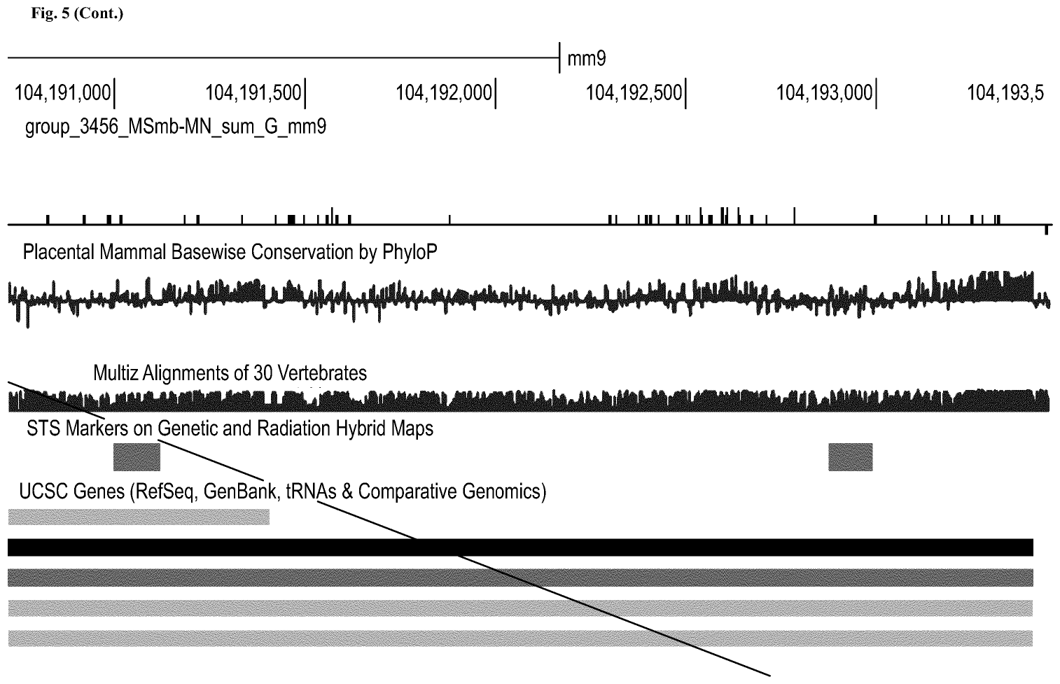

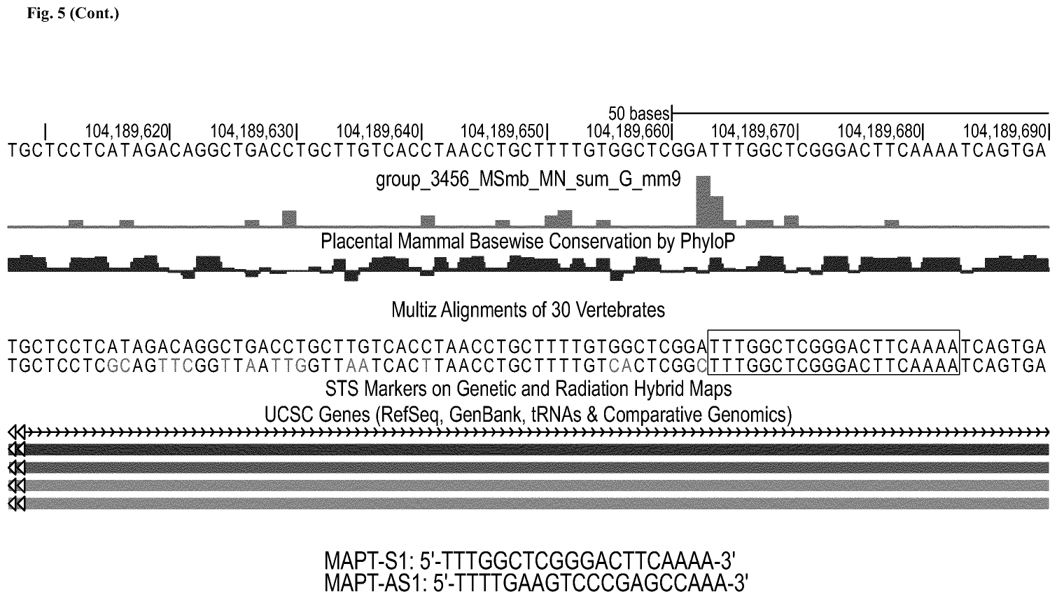

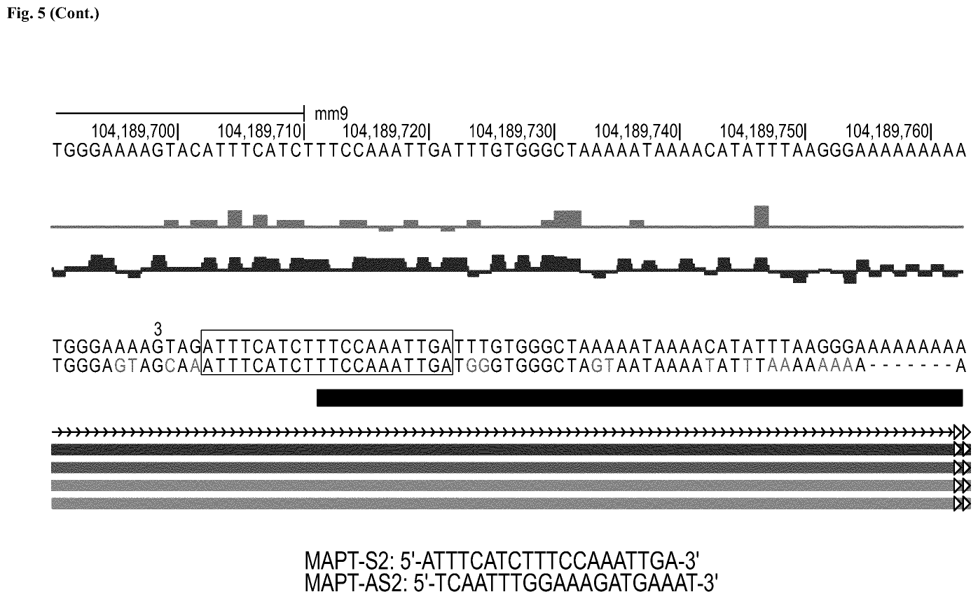

ASOs can be generated that bind to these two mRNA sites to prevent the association with the hnRNP R binding protein. FIG. 5 shows the design of ASOs to inhibit hnRNP R binding to MAPT.

Two exemplary ASOs that bind to the MAPT mRNA sites and will prevent the association with the hnRNP R binding protein are: MAPT-ASO1: 5′-TTTTGAAGTCCCGAGCCAAA-3′ and MAPT-ASO2: 5′-TCAATTTGGAAAGATGAAAT-3′

### Example 2

Blocking axonal Mapt transport was investigated for reduction of axonal tau protein production, providing a therapeutic strategy for protection of neurons against NFT formation and spreading.

In this Example, it is demonstrated that the 3′ UTR of Mapt contains binding sites for the RNA-binding protein hnRNP R, and that loss of hnRNP R selectively abolishes axonal Mapt translocation and reduces axonal tau without affecting total levels of Mapt and tau. Antisense oligonucleotides (ASOs) were designed for blocking the association between hnRNP R and Mapt, and a similar reduction in axonal Mapt and tau in neurons treated with these ASOs was observed. Thus, ASO-mediated depletion of axonal tau could be a therapeutic option for treatment of AD and other tauopathies.

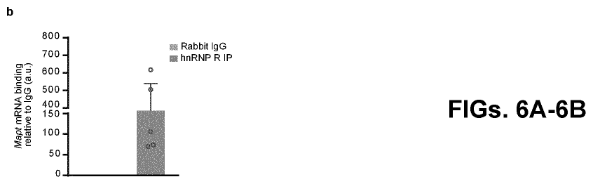

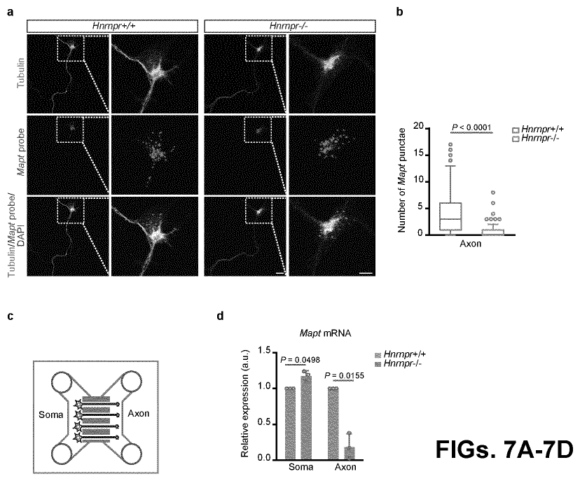

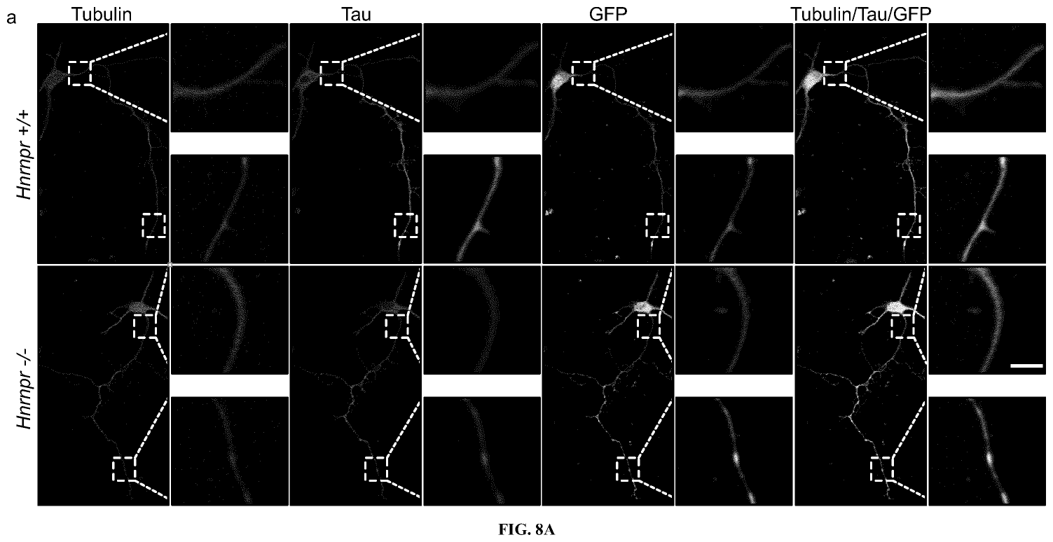

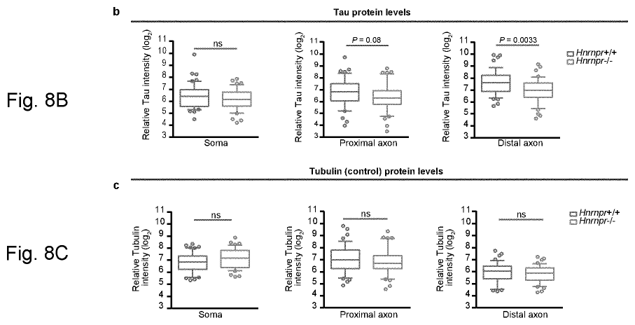

In this Example, individual nucleotide resolution and crosslinking immunoprecipitation (iCLIP) for hnRNP R in primary mouse embryonic motoneurons was performed. Visual inspection of the Mapt transcript identified hnRNP R iCLIP hits in the 3′ UTR (FIG. 6A). RNA immunoprecipitation using an antibody against hnRNP R confirmed its association with Mapt (FIG. 6B). Whether hnRNP R regulates the subcellular distribution of Mapt was also investigated. Reduced axonal Mapt levels were detectable in motoneurons derived from hnRNP R knockout mice (Hnrnpr−/−) relative to +/+ motoneurons by fluorescent in situ hybridization (FISH) (FIGS. 7A-7B). Axonal but not somatodendritic levels of Mapt mRNA were also reduced in Hnrnpr−/− compared to +/+ motoneurons cultured in microfluidic chambers for 6 days in vitro (DIV) (FIGS. 7C-7D). Immunostaining for tau revealed lower tau protein levels in axons but not cell bodies of Hnrnpr−/− motoneurons (FIGS. 8A-8C). Thus, hnRNP R regulates axonal tau levels through translocation of Mapt.

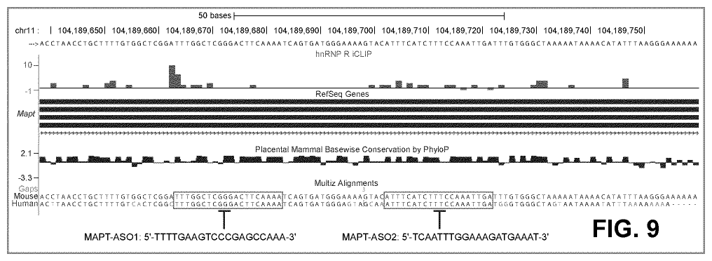

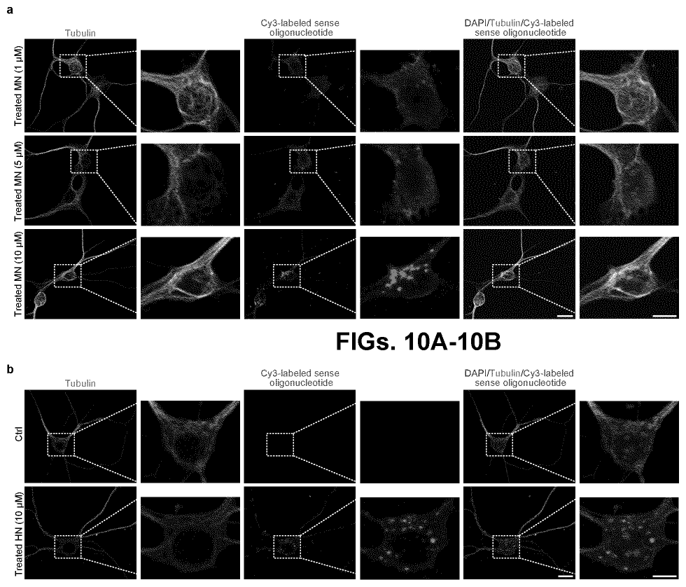

Continuing in this Example, two 2′-O-methyl-and phosphorothiate-modified ASOs (MAPT-ASO1 and 2) were designed complementary to the Mapt 3′ UTR regions with hnRNP R iCLIP hits for blocking the association between hnRNP R and Mapt (FIG. 9). Binding regions that were conserved or substantially conserved between mouse and human were selected. For optimization of uptake conditions, a Cy3-labeled sense oligonucleotide was used. Efficient uptake was observed by incubating motoneurons (FIG. 10A) or hippocampal neurons (FIG. 10B) with 10 μM oligonucleotide.

In the present example, Example 2, motoneurons treated with MAPT-ASO1 or MAPT-ASO2 and cultured for 6 DIV showed reduced axonal Mapt mRNA levels compared to untreated motoneurons as detected by FISH (FIGS. 11A-11C). A similar reduction in axonal Mapt was also detectable in cultured hippocampal neurons (FIGS. 11D-11F). Importantly, Mapt levels in the cell bodies of MAPT-ASO-treated neurons were unchanged (FIGS. 11B, 11E).

Next, whether MAPT-ASO-mediated depletion of axonal Mapt can also downregulate tau protein in axons was investigated. Given the relatively long half-life of tau, tau was assessed by immunostaining in MAPT-ASO-treated and untreated motoneurons cultured for 11 DIV. Following MAPT-ASO treatment, tau was reduced in axons but not cell bodies of motoneurons (FIGS. 12A-12D). The axonal tau reduction was stronger for MAPT-ASO2 compared to MAPT-ASO1 (FIG. 12D). Together, these data indicate that tau protein levels can be selectively reduced in axons by ASOs blocking the hnRNP R binding sites in the Mapt 3′ UTR.

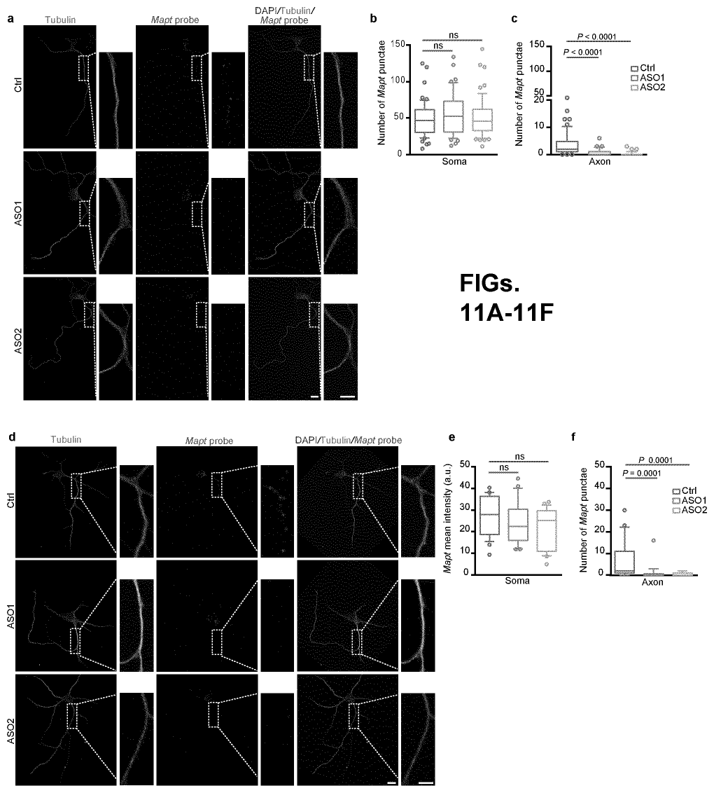

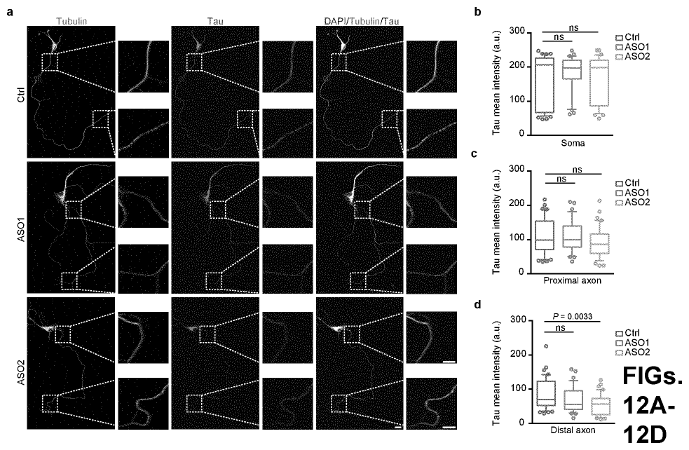

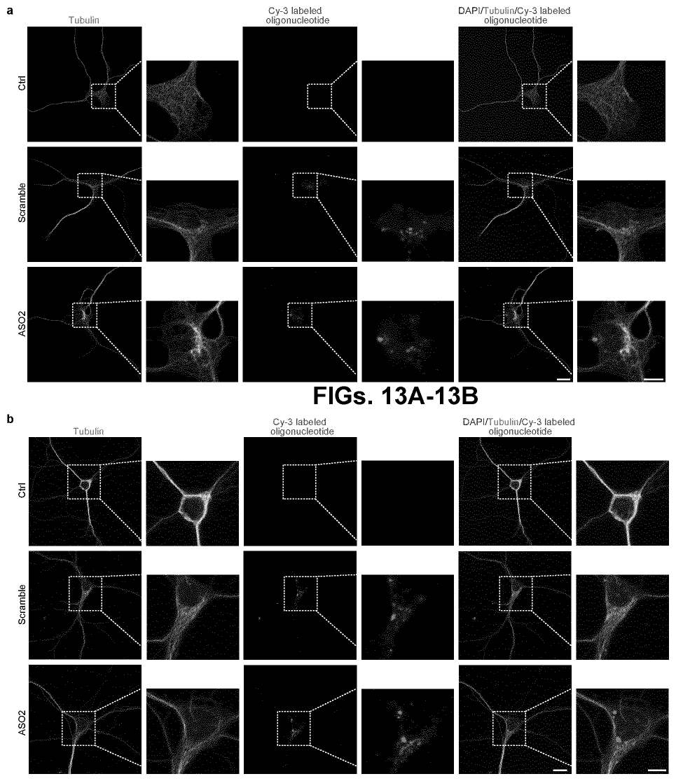

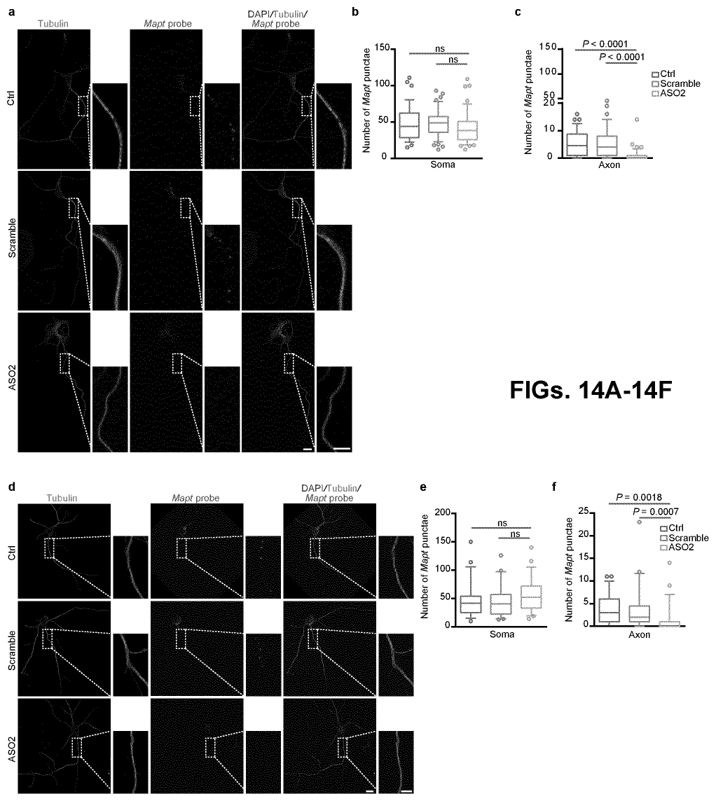

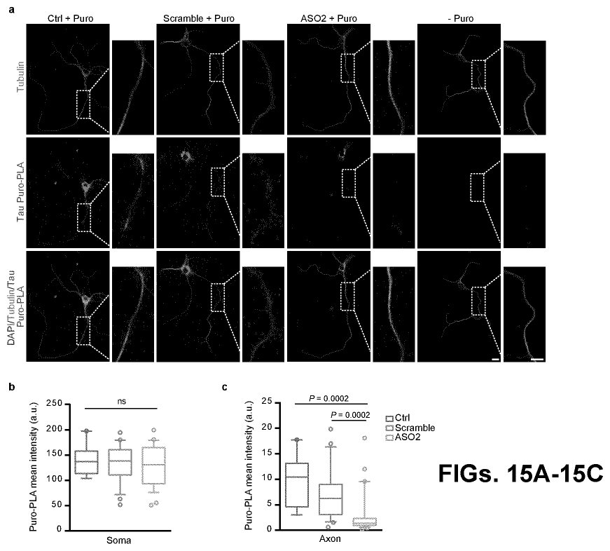

Having demonstrated that MAPT-ASO2 more efficiently reduces axonal tau relative to MAPT-ASO1, MAPT-ASO2 was used and compared to a scrambled version of it (MAPT-ASO2Scr) as additional control. At 10 μM, Cy3-labeled MAPT-ASO2 and Scr were efficiently taken up by motoneurons (FIG. 13A) and hippocampal neurons (FIG. 13B). Compared to Scr, MAPT-ASO2 significantly downregulated axonal Mapt levels in motoneurons (FIGS. 14A-14C) and hippocampal neurons (FIGS. 14D-14F). Puro-PLA was then used to assess the axonal translation of tau. Motoneurons treated with MAPT-ASO2 revealed a reduced Puro-PLA signal for tau in axons compared to Scr-treated and untreated motoneurons (FIGS. 15A-15C). Tau synthesis in cell bodies was unaffected by MAPT-ASO2 treatment (FIG. 15B).

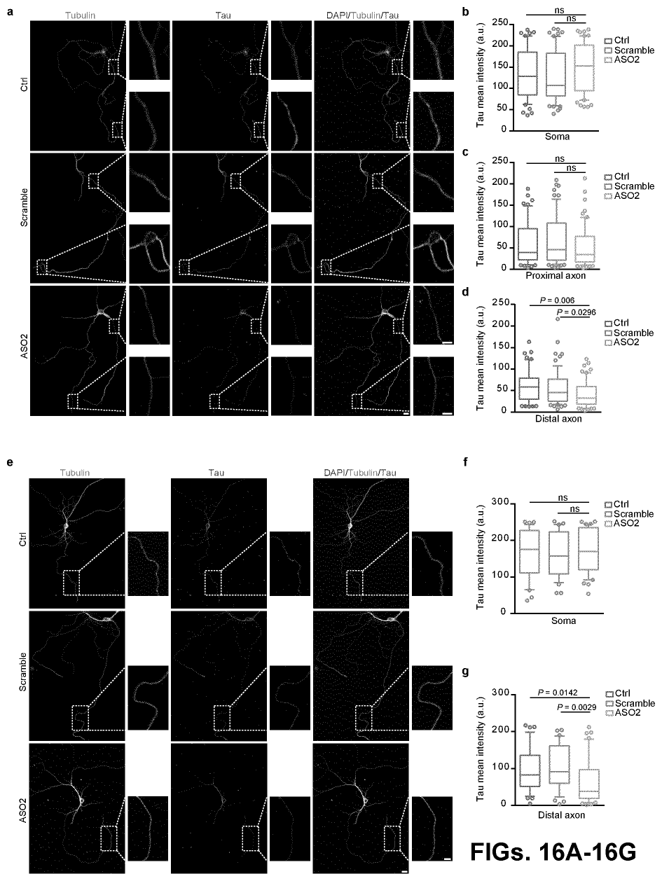

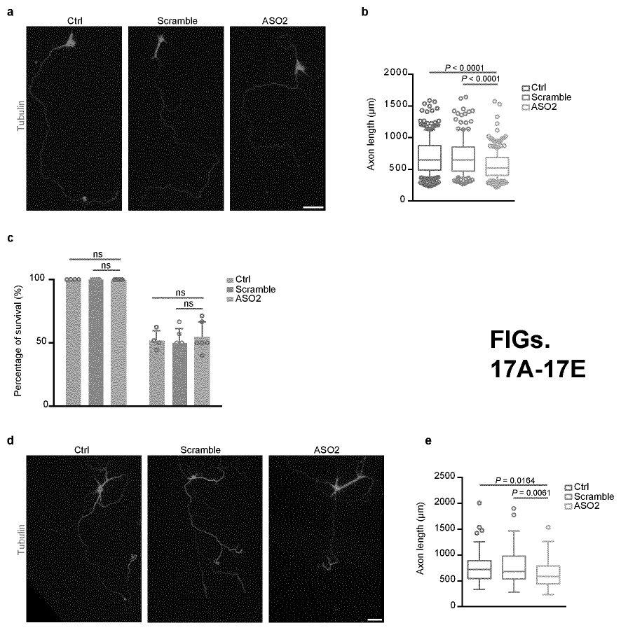

Likewise, tau protein levels were reduced in axons of MAPT-ASO2-treated motoneurons (FIGS. 16A-16D) and hippocampal neurons (FIGS. 16E-16G) compared Scr-treated neurons. In agreement with a function of tau in axon growth, axon lengths of motoneurons subjected to MAPT-ASO2 treatment were reduced compared to Scr treatment while survival was unaffected (FIGS. 17A-17C). Reduced axon growth was also detectable for hippocampal neurons exposed to MAPT-ASO2 (FIGS. 17D-17E). Thus, MAPT-ASO2 treatment can reduce axonal levels of Mapt, resulting in less axonal tau due to lowered local translation.

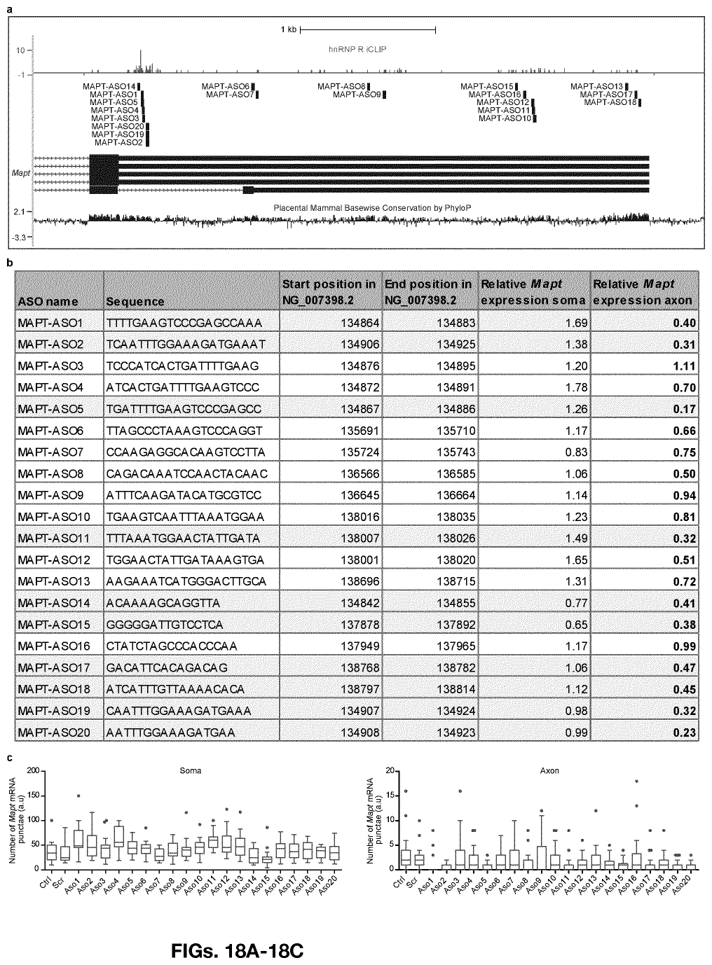

Additional MAPT-ASOs were designed along the Mapt 3′ UTR in regions that contain hnRNP R iCLIP hits and that are conserved between mouse and human (FIGS. 18A-18B). These ASOs were screened by fluorescene in situ hybridization (FISH) in hippocampal neurons for their potential to reduce axonal Mapt mRNA levels. Several candidates were identified that lowered axonal Mapt levels >50% in MAPT-ASO-treated relative to untreated hippocampal neurons (FIGS. 18B-18C). Two of these MAPT-ASOs (19 and 20) were shortened versions of MAPT-ASO2 with a length of 18 and 16 nucleotides, respectively.

Tau reduction has emerged as a promising therapeutic strategy for treatment of AD and other tauopathies. However, prolonged depletion of tau might be detrimental, warranting the development of more targeted approaches to selectively prevent tau elevation and NFT formation in axons. The strategy demonstrated here makes use of the hnRNP R-dependent axonal transport of Mapt mRNA and its local translation into tau protein. By blocking the interaction between Mapt and hnRNP R, a reduction in axonal tau levels can be achieved without affecting tau levels in the soma (FIG. 4). If administered sufficiently early in the course of the disease, the MAPT-ASOs described here could be useful for limiting the initiation and spreading of tau pathology in AD.

### Example 3

This example is directed to a method whereby the number of SPs and NFTs is reduced through depleting the RNA-binding protein hnRNP R. This method is based on the observation that, when hnRNP R is missing, brains of 5xFAD mice, an AD mouse model overexpressing mutant human amyloid precursor protein (APP) and presenilin 1 (PS 1), exhibit reduced numbers of SPs.

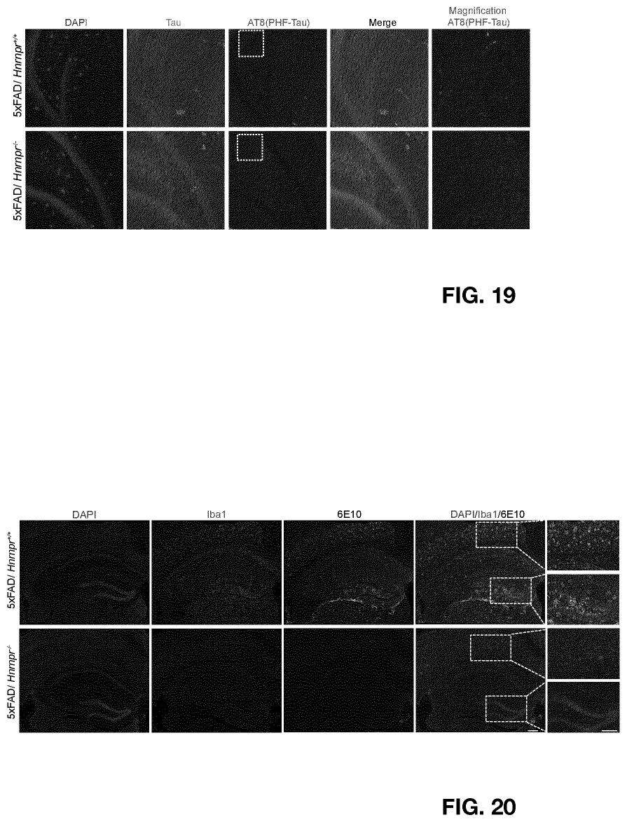

As shown in the images of FIG. 19, at 9 months of age, 5xFAD mice show widespread deposition of SPs in cortex and hippocampus, accompanied by activated microglia revealed by lba1 immunostaining. However, 5xFAD mice homozygous for a hnRNP R knockout allele (5xFAD; HInrnpr−/−), have reduced SP deposition and less microglial activation.

As shown in the images of FIG. 20, lack of hnRNP R also led to lower amounts of hyperphosphorylated tau, the main component of NFTs, in these brain regions.

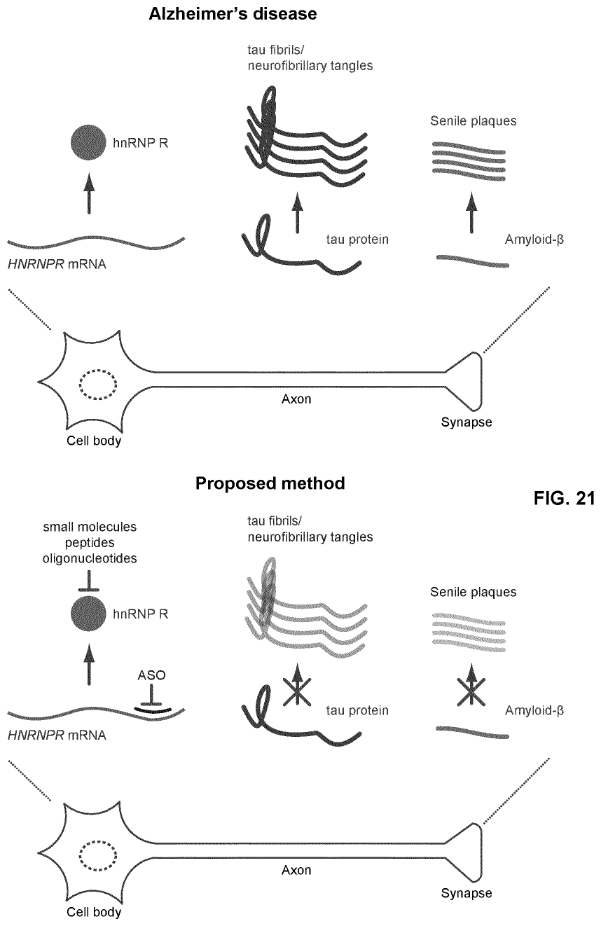

These results support that depletion of hnRNP R through antisense oligonucleotide-mediated degradation of its mRNA or inhibition of hnRNP R through small molecules, peptides and oligonucleotides can be used to lower the amounts of SPs and NFTs, representing a therapeutic option for the treatment of AD. This mechanism is shown in illustrated form in FIG. 21.

This detailed description is to be understood as being in every respect illustrative and exemplary, but not restrictive, and the scope of the inventive concept disclosed herein should be interpreted according to the full breadth permitted by the patent laws. It is to be understood that the embodiments shown and described herein are only illustrative of the principles of the inventive concept and that various modifications may be implemented by those skilled in the art without departing from the scope and spirit of the inventive concept. Those skilled in the art could implement various other feature combinations without departing from the scope and spirit of the inventive concept.

1. A method for reducing axonal tau protein comprising:
inhibiting the binding between MAPT mRNA and hnRNP R, wherein antisense oligonucleotide binding molecules are bound to binding sites of the MAPT mRNA, wherein the antisense oligonucleotide is selected from the group consisting of (i) MAPT-ASO19, and (ii) MAPT-ASO20.

2. The method of claim 1, wherein the antisense oligonucleotide is MAPT-ASO19.

3. The method of claim 1, wherein the antisense oligonucleotide is MAPT-ASO20.

4-11. (canceled)

12. A method for reducing hnRNP R protein levels in a subject, the method comprising:
administering an antisense oligonucleotide to the subject, wherein the antisense oligonucleotide is configured to reduce hnRNP R mRNA levels, wherein the antisense oligonucleotide is selected from the group consisting of (i) MAPT-ASO19, and (ii) MAPT-ASO20.

13. (canceled)

14. A method for treating a subject with Alzheimer's disease, the method comprising:
administering one or more of:
an antisense oligonucleotide to the subject, wherein the antisense oligonucleotide is configured to reduce hnRNP R mRNA levels, wherein the antisense oligonucleotide is selected from the group consisting of (i) MAPT-ASO19, and (ii) MAPT-ASO20.

15. A pharmaceutical composition for treating Alzheimer's disease, the composition comprising:
an antisense oligonucleotide is selected from the group consisting of (i) MAPT-ASO19, and (ii) MAPT-ASO20 or derivatives thereof; and
a pharmaceutically acceptable carrier or excipient.

16-17. (canceled)

18. The method of claim 12, wherein the antisense oligonucleotide is MAPT-ASO19.

19. The method of claim 12, wherein the antisense oligonucleotide is MAPT-ASO20.

20. The method of claim 14, wherein the antisense oligonucleotide is MAPT-ASO19.

21. The method of claim 14, wherein the antisense oligonucleotide is MAPT-ASO20.

22. The method of claim 15, wherein the antisense oligonucleotide is MAPT-ASO19.

23. The method of claim 15, wherein the antisense oligonucleotide is MAPT-ASO20.
